(19) United States
(12) Patent Application Publication
(10) Pub. No.: US 2023/0290468 A1
(43) Pub. Date: Sep. 14, 2023

(54) TECHNOLOGIES FOR PROVIDING ENHANCED PAIN MANAGEMENT

(71) Applicant: Hill-Rom Services, Inc., Batesville, IN (US)

(72) Inventors: Susan Amanda Kayser, Batesville, IN (US); Rachel Lynn Williamson, Batesville, IN (US); Angela Christine Murray, Batesville, IN (US); Kelli F. Rempel, Chapel Hill, NC (US); Sinan Batman, Batesville, IN (US); Georg Köllner, Batesville, IN (US); Michael Scott Hood, Batesville, IN (US); Lori Ann Zapfe, Milroy, IN (US); Gene J. Wolfe, Pittsford, NY (US)

(21) Appl. No.: 18/175,622

(22) Filed: Feb. 28, 2023

Related U.S. Application Data
(60) Provisional application No. 63/318,828, filed on Mar. 11, 2022.

(51) Int. Cl. G16H 20/10; A61B 5/0205; A61B 5/00

(52) U.S. Cl. CPC G16H 20/10; A61B 5/0205; A61B 5/746

(57) ABSTRACT

A compute device may include circuitry configured to obtain patient state data that may be indicative of a heart rate of the patient or a respiration rate of the patient. The circuitry may also be configured to obtain patient medication data indicative of a schedule for administration of pain medication to the patient. Further, the circuitry may be configured to determine whether a trend in the patient state data satisfies a predefined condition, determine whether the patient medication data indicates that the patient is due for administration of pain medication within a predefined time period, determine, in response to a determination that the trend in the patient state data satisfies the predefined condition and a determination that the patient is due for administration of pain medication within the predefined time period, that the patient is experiencing pain, and produce an alert signal that the patient is in pain.

### CROSS-REFERENCE TO RELATED APPLICATION

This application claims the benefit, under 35 U.S.C. § 119(e), of U.S. Provisional Patent Application No. 63/318,828, filed Mar. 11, 2022, the entirety of which is hereby expressly incorporated by reference herein.

### BACKGROUND

The present disclosure relates to pain management for patients receiving healthcare services, and more particularly to the measurement of pain experienced by such patients and coordinating care services provided to the patient based on the measured pain.

Pain is known in the field of medical care to be difficult to measure. In conventional medical care settings, pain assessments rely on a patient to verbally report their pain, such as on a scale of zero to ten, with zero representing no pain and ten representing excruciating pain. By their nature, such pain assessments may provide inconsistent results that can vary based on how, when, and by who the assessment was conducted, in addition the patient's mental state, bias, and training. Furthermore, in instances in which the patient is not conscious or cannot communicate (e.g., during surgery, babies, elderly patients, intubated patients, patients with dementia or speech impairment, etc.), question and answer-based approaches are not feasible. Additionally, the experience of pain is subjective and one patient may experience a different level of pain than another patient under similar circumstances. As such, patients and caregivers may be unable to accurately predict the level of pain that a patient will experience in a medical care process, such as a surgical procedure, a pre-surgical procedure, a post-surgical procedure, physical therapy, or the like. With an inability to accurately assess a patient's pain, complications from underestimating the pain, such as not mobilizing a patient for physical therapy, or overestimating the pain, such as over-administration of pain medication resulting in respiratory distress, may result.

### SUMMARY

The present application discloses one or more of the features recited in the appended claims and/or the following features which, alone or in any combination, may comprise patentable subject matter:

According to an aspect of the present disclosure, a compute device (e.g., a computer) may include circuitry configured to obtain patient state data. The patient state data may be indicative of a present state of a patient detected by one or more patient monitor devices. The patient state data may include at least one of heart rate data indicative of a heart rate of the patient or respiration rate data indicative of a respiration rate of the patient. The circuitry may be further configured to obtain patient medication data (e.g., from an electronic medical records system or other source, such as an intravenous pump). The patient medication data may be indicative of a schedule for administration of pain medication to the patient. Additionally, the circuitry may be configured to determine whether a trend in the patient state data satisfies a predefined condition. Further, the circuitry of the compute device may be configured to determine whether the patient medication data indicates that the patient is due for administration of pain medication within a predefined time period. Additionally, the circuitry may be configured to determine, in response to a determination that the trend in the patient state data satisfies the predefined condition and a determination that the patient is due for administration of pain medication within the predefined time period, that the patient is experiencing pain. The circuitry may be further configured to produce, in response to a determination that the patient is experiencing pain, an alert signal.

In some embodiments, the circuitry may be configured to administer, in response to a determination that the patient is experiencing pain, pain medication to the patient using a pain medication administration device. The circuitry may be configured to produce an alert signal that includes an audible alert, an alert on a screen, a nurse call signal, and/or a message to a caregiver mobile device. Additionally or alternatively, the circuitry may be configured to determine a pain medication administration time that may be indicative of when the patient was last administered pain medication, determine a decline in the respiration rate of the patient over a predefined time period after the pain medication administration time, and determine whether the decline satisfies a reference decline that may be indicative of opioid induced respiratory distress.

In some embodiments, the circuitry may be further configured to provide, in response to a determination that the decline satisfies the reference decline, a notification to a caregiver that may indicate that the patient is experiencing opioid induced respiratory distress. The circuitry may be configured such that determining whether the decline in the respiration rate satisfies a reference decline includes determining whether the respiration rate has decreased by at least five breaths per minute or 30% of an initial respiration rate within a period of four hours after the pain medication administration time.

In some embodiments, in determining whether a trend in the patient state data satisfies a predefined condition, the circuitry may be configured to determine whether the respiration rate of the patient has increased by five breaths per minute or 30% in less than three hours.

The circuitry of the compute device in some embodiments may be configured such that determining whether a trend in the patient state data satisfies a predefined condition includes determining whether the heart rate of the patient has increased by 15 beats per minute or 30% in less than three hours. In some embodiments, the circuitry may be configured such that determining whether the patient medication data indicates that the patient is due for administration of pain medication within a predefined time period includes determining whether the patient is due for administration of pain medication within 15 minutes.

The circuitry of the compute device may be further configured to obtain patient state data indicative of movement of the patient. Additionally, the circuitry may be configured such that determining whether a trend in the patient state data satisfies a predefined condition incudes determining whether the movement of the patient has increased and determining whether the respiration rate of the patient has increased by five breaths per minute or 30% in less than three hours or the heart rate of the patient has increased by 15 beats per minute or 30% in less than three hours.

In some embodiments, the circuitry is configured to obtain movement magnitude data that may be indicative of magnitudes of movements of the patient and movement frequency data that may be indicative of a frequency of movements of the patient. The circuitry may be further configured to determine whether at least one of the magnitudes of the movements of the patient or the frequency of the movements of the patient has increased. The circuitry may be additionally or alternatively configured to obtain patient movement data from at least one of a set of load cells in a patient support apparatus, an image capture device directed at the patient, or a wearable device that may be worn by the patient. In some embodiments, the circuitry may be configured to obtain heart rate variability data that may be indicative of lengths of time between heart beats of the patient, as part of the patient state data.

In another aspect of the present disclosure, a method may include obtaining, by a compute device, patient state data that may be indicative of a present state of a patient that may be detected by one or more patient monitor devices. The patient state data may include at least one of heart rate data that may be indicative of a heart rate of the patient or respiration rate data that may be indicative of a respiration rate of the patient. Additionally, the method may include obtaining, by the compute device, patient medication data that may be indicative of a schedule for administration of pain medication to the patient. Further, the method may include determining, by the compute device, whether a trend in the patient state data satisfies a predefined condition. Additionally, the method may include determining, by the compute device, whether the patient medication data indicates that the patient is due for administration of pain medication within a predefined time period. The method may also include determining, by the compute device and in response to a determination that the trend in the patient state data satisfies the predefined condition and a determination that the patient is due for administration of pain medication within the predefined time period, that the patient is experiencing pain. Further, the method may include producing, by the compute device and in response to a determination that the patient is experiencing pain, an alert signal.

The method may additionally include administering, by the compute device and in response to a determination that the patient is experiencing pain, pain medication to the patient using a pain medication administration device. In some embodiments, producing an alert signal includes producing an audible alert, an alert on a screen, a nurse call signal, and/or a message to a caregiver mobile device. The method may additionally or alternatively include determining, by the compute device, a pain medication administration time indicative of when the patient was last administered pain medication. In addition, the method may include determining, by the compute device, a decline in the respiration rate of the patient over a predefined time period after the pain medication administration time. Further, the method may include determining, by the compute device, whether the decline satisfies a reference decline that may be indicative of opioid induced respiratory distress.

In some embodiments, the method may include providing, by the compute device and in response to a determination that the decline satisfies the reference decline, a notification to a caregiver that the patient is experiencing opioid induced respiratory distress. Determining whether the decline in the respiration rate satisfies a reference decline may, in some embodiments of the method, include determining whether the respiration rate has decreased by at least five breaths per minute or 30% of an initial respiration rate within a period of four hours after the pain medication administration time.

Determining whether a trend in the patient state data satisfies a predefined condition may include determining whether the respiration rate of the patient has increased by five breaths per minute or 30% in less than three hours. In some embodiments, determining whether a trend in the patient state data satisfies a predefined condition may include determining whether the heart rate of the patient has increased by 15 beats per minute or 30% in less than three hours.

Determining whether the patient medication data indicates that the patient is due for administration of pain medication within a predefined time period may, in some embodiments, include determining whether the patient is due for administration of pain medication within 15 minutes or a different configurable amount of time. Some embodiments of the method may additionally include obtaining, by the compute device, patient state data that may be indicative of movement of the patient. Additionally, determining whether a trend in the patient state data satisfies a predefined condition may include determining, by the compute device, whether the movement of the patient has increased and determining, by the compute device, whether the respiration rate of the patient has increased by five breaths per minute or 30% in less than three hours or the heart rate of the patient has increased by 15 beats per minute or 30% in less than three hours.

In some embodiments, obtaining patient state data indicative of movement of the patient may include obtaining movement magnitude data that may be indicative of magnitudes of movements of the patient and movement frequency data that may be indicative of a frequency of movements of the patient. Determining whether the movement of the patient has increased may include determining whether at least one of the magnitudes of the movements of the patient or the frequency of the movements of the patient has increased.

Obtaining patient state data indicative of movement of the patient may include obtaining patient movement data from at least one of a set of load cells in a patient support apparatus, an image capture device directed at the patient, or a wearable device that may be worn by the patient. In some embodiments, obtaining patient state data may include obtaining heart rate variability data that may be indicative of lengths of time between heart beats of the patient.

In another aspect of the invention, one or more computer-readable storage media may include a plurality of instructions that, when executed, may cause a compute device to obtain patient state data. The patient state data may be indicative of a present state of a patient that may be detected by one or more patient monitor devices. The patient state data may include at least one of heart rate data that may be indicative of a heart rate of the patient or respiration rate data that may be indicative of a respiration rate of the patient. The instructions may additionally cause the compute device to obtain patient medication data that may be indicative of a schedule for administration of pain medication to the patient. In addition, the instructions may cause the compute device to determine whether a trend in the patient state data satisfies a predefined condition, determine whether the patient medication data indicates that the patient is due for administration of pain medication within a predefined time period, and determine, in response to a determination that the trend in the patient state data satisfies the predefined condition and a determination that the patient is due for administration of pain medication within the predefined time period, that the patient is experiencing pain. The instructions may additionally cause the compute device to produce, in response to a determination that the patient is experiencing pain, an alert signal.

In some embodiments, the instructions may cause the compute device to administer, in response to a determination that the patient is experiencing pain, pain medication to the patient using a pain medication administration device. The instructions may further cause the compute device to produce, as the alert signal, an audible alert, an alert on a screen, a nurse call signal, and/or a message to a caregiver mobile device. In some embodiments, the instructions may cause the compute device to determine a pain medication administration time indicative of when the patient was last administered pain medication. The instructions may further cause the compute device to determine a decline in the respiration rate of the patient over a predefined time period after the pain medication administration time and determine whether the decline satisfies a reference decline indicative of opioid induced respiratory distress.

In some embodiments, the instructions may further cause the compute device to provide, in response to a determination that the decline satisfies the reference decline, a notification to a caregiver that the patient is experiencing opioid induced respiratory distress. The instructions may cause the compute device, in some embodiments, to determine whether the respiration rate has decreased by at least five breaths per minute or 30% of an initial respiration rate within a period of four hours after the pain medication administration time. In some embodiments, the instructions may cause the compute device to determine whether the respiration rate of the patient has increased by five breaths per minute or 30% in less than three hours.

In some embodiments, the instructions may cause the compute device to determine whether a trend in the patient state data satisfies a predefined condition by determining whether the heart rate of the patient has increased by 15 beats per minute or 30% in less than three hours. The instructions may also cause the compute device to determine whether the patient medication data indicates that the patient is due for administration of pain medication within a predefined time period by determining whether the patient is due for administration of pain medication within 15 minutes or a different configurable amount of time. In some embodiments, the instructions may further cause the compute device to obtain patient state data that may be indicative of movement of the patient. The instructions may additionally cause the compute device to determine whether a trend in the patient state data satisfies a predefined condition by determining whether the movement of the patient has increased and determining whether the respiration rate of the patient has increased by five breaths per minute or 30% in less than three hours or the heart rate of the patient has increased by 15 beats per minute or 30% in less than three hours.

In some embodiments, the instructions may cause the compute device to obtain movement magnitude data that may be indicative of magnitudes of movements of the patient and movement frequency data that may be indicative of a frequency of movements of the patient. The instructions may further cause the compute device to determine whether at least one of the magnitudes of the movements of the patient or the frequency of the movements of the patient has increased. Obtaining patient state data indicative of movement of the patient may, in some embodiments, include obtaining patient movement data from at least one of a set of load cells in a patient support apparatus, an image capture device directed at the patient, or a wearable device that may be worn by the patient. In some embodiments, the instructions may cause the compute device to obtain heart rate variability data indicative of lengths of time between heart beats of the patient as part of the patient state data.

In another aspect of the present disclosure, a compute device includes circuitry configured to apply a pain stimulus to a patient. The circuitry may also be configured to obtain patient state data that may be indicative of a present state of the patient detected by one or more patient monitor devices in response to the pain stimulus. Additionally, the circuitry may be configured to train a machine learning model to determine a pain sensitivity of the patient as a function of the pain stimulus and the patient state data. Further the circuitry may be configured to produce, based on the determined pain sensitivity of the patient, information that is usable to manage pain in association with a medical care process.

In some embodiments, the circuitry may be further configured to obtain patient context data. The patient context data may be indicative of a medical context of the patient, including at least one of a stage of a physical therapy program associated with the patient, a hormonal level of the patient, a sleep quality of the patient, a history of movement of the patient, chronic pain experienced by the patient, or a pain medication schedule of the patient. Additionally, the circuitry may be configured to train the machine learning model to determine a pain sensitivity of the patient further as a function of the patient context data. The circuitry may be further configured to provide a notification to adjust at least one of a physical therapy program or a pain medication schedule associated with the patient to manage the pain.

The circuitry, in some embodiments, may be configured to obtain audio data indicative of words or sounds associated with the patient or image data (e.g., image(s) of the patient grimacing, wincing, etc., to be used in a facial recognition process) as part of the patient state data. In some embodiments, the circuitry may be configured to determine, with the machine learning model, whether the patient is presently experiencing pain. The circuitry may be further configured to provide a report that may be indicative of the determination of whether the patient is presently experiencing pain. In some embodiments, the circuitry may be further configured to send the report to an electronic medical records system. The circuitry in some embodiments may be further configured to provide a report that includes an indication of whether the patient has an underlying health issue that contributes to pain.

Additionally or alternatively, the circuitry may be further configured to provide a report that may be indicative of pain to be expected in association with a medical care process. In some embodiments, the circuitry may be configured to apply an electrical stimulus to the patient or a thermal stimulus to the patient. The circuitry of the compute device may be configured to obtain data indicative of a change in electrical impedance of the patient as part of the patient state data. In some embodiments, the circuitry may be configured to apply the pain stimulus to the patient by causing the patient to perform a movement known to potentially induce pain. The movement may be a movement associated with a physical therapy program.

In another aspect of the present disclosure, a method may include applying, by a compute device, a pain stimulus to a patient. The method may also include obtaining, by the compute device, patient state data that may be indicative of a present state of the patient detected by one or more patient monitor devices in response to the pain stimulus. Additionally, the method may include training, by the compute device, a machine learning model to determine a pain sensitivity of the patient as a function of the pain stimulus and the patient state data. Further, the method may include producing, by the compute device and based on the determined pain sensitivity of the patient, information that is usable to manage pain in association with a medical care process.

The method may also include obtaining, by the compute device, patient context data that may be indicative of a medical context of the patient. The patient context data may include at least one of a stage of a physical therapy program associated with the patient, a hormonal level of the patient, a sleep quality of the patient, a history of movement of the patient, chronic pain experienced by the patient, or a pain medication schedule of the patient. The method may also include training, by the compute device, the machine learning model to determine a pain sensitivity of the patient further as a function of the patient context data.

In some embodiments, the method additionally includes providing, by the compute device, a notification to adjust at least one of a physical therapy program or a pain medication schedule associated with the patient to manage the pain. Obtaining patient state data may further include obtaining audio data that may be indicative of words or sounds associated with the patient (e.g., groaning, crying, moaning, words or phrases such as “I'm in pain,” “that hurts,” “ouch,” etc.) or image data that may be indicative of facial expressions indicative of pain (e.g., grimacing, wincing, etc.). In some embodiments, the method also includes determining, by the compute device and with the machine learning model, whether the patient is presently experiencing pain. The method may also include providing, by the compute device, a report indicative of the determination of whether the patient is presently experiencing pain.

In some embodiments, the method may include sending, by the compute device, the report to an electronic medical records system. The method may additionally or alternatively include providing, by the compute device, a report that may include an indication of whether the patient has an underlying health issue that contributes to pain. In some embodiments, the method also includes providing, by the compute device, a report that may be indicative of pain to be expected in association with a medical care process. Applying the pain stimulus to the patient may include applying an electrical stimulus to the patient or a thermal stimulus to the patient. Further, obtaining patient state data may include obtaining data indicative of a change in electrical impedance of the patient. In some embodiments, applying the pain stimulus to the patient includes causing the patient to perform a movement known to potentially induce pain. Applying the pain stimulus to the patient may include causing the patient to perform a movement associated with a physical therapy program.

In another aspect of the disclosure, one or more computer-readable storage media may include a set of instructions that, when executed, may cause a compute device to apply a pain stimulus to a patient. The instructions may also cause the compute device to obtain patient state data that may be indicative of a present state of the patient detected by one or more patient monitor devices in response to the pain stimulus. The instructions may also cause the compute device to train a machine learning model to determine a pain sensitivity of the patient as a function of the pain stimulus and the patient state data. Additionally, the instructions may cause the compute device to produce, based on the determined pain sensitivity of the patient, information that is usable to manage pain in association with a medical care process.

In some embodiments, the instructions may further cause the compute device to obtain patient context data that may be indicative of a medical context of the patient. The patient context data may include at least one of a stage of a physical therapy program associated with the patient, a hormonal level of the patient, a sleep quality of the patient, a history of movement of the patient, chronic pain experienced by the patient, or a pain medication schedule of the patient. The instructions may additionally cause the compute device to train the machine learning model to determine a pain sensitivity of the patient further as a function of the patient context data.

The instructions may, in some embodiments, cause the compute device to provide a notification to adjust at least one of a physical therapy program or a pain medication schedule associated with the patient to manage the pain. In some embodiments, the instructions may cause the compute device to obtain audio data that may be indicative of words or sounds associated with the patient or image data (e.g., image(s) of the patient grimacing, wincing, etc.).

In some embodiments, the instructions may further cause the compute device to determine, with the machine learning model, whether the patient is presently experiencing pain. The instructions may additionally or alternatively cause the compute device to provide a report that may be indicative of the determination of whether the patient is presently experiencing pain. In some embodiments, the instructions may further cause the compute device to send the report to an electronic medical records system. The instructions may also cause the compute device to provide a report that may include an indication of whether the patient has an underlying health issue that contributes to pain.

The one or more computer-readable storage media may also have instructions that may cause the compute device to provide a report indicative of pain to be expected in association with a medical care process. In some embodiments, the instructions may cause the compute device to apply an electrical stimulus to the patient or a thermal stimulus to the patient. The instructions may cause the compute device to obtain data indicative of a change in electrical impedance of the patient. In some embodiments, the instructions may cause the compute device to apply the pain stimulus to the patient by causing the patient to perform a movement known to potentially induce pain. The movement may be associated with a physical therapy program.

In another aspect of the disclosure, a compute device may include circuitry configured to obtain patient state data. The patient state data may be indicative of a present state of a patient and may include a heart rate of the patient and/or a respiration rate of the patient. The compute device may also include circuitry that may be configured to determine whether a change in patient state data satisfies a reference change. Additionally, the compute device may include circuitry that may be configured to determine, in response to a determination that the change in the patient state data satisfies the reference change, that the patient is experiencing pain. Further, the compute device may include circuitry that may be configured to produce a notification to another device communicatively connected to the compute device, indicating that the patient is experiencing pain.

In some embodiments, the compute device may additionally include circuitry that may be configured to obtain patient medication data from an intravenous pump associated with the patient. Further, the compute device may include circuitry that may be configured to determine, further as a function of the patient medication data, whether the patient is experiencing pain. In some embodiments, the compute device may include circuitry that may be configured to obtain image data that may be indicative of a facial expression of the patient. The compute device may also include circuitry configured to determine, further as a function of the obtained image data, whether the patient is experiencing pain. The compute device, in some embodiments, may include circuitry that may be configured to obtain audio data that may be indicative of one or more sounds produced by the patient. Additionally, the compute device may include circuitry that may be configured to determine, further as a function of the obtained audio data, whether the patient is experiencing pain.

In another aspect of the present disclosure, a method may include obtaining, by a compute device, patient state data that may be indicative of a present state of a patient. The patient state data may include a heart rate of the patient and/or a respiration rate of the patient. Additionally, the method may include determining, by the compute device, whether a change in patient state data satisfies a reference change. Further, the method may include determining, by the compute device and in response to a determination that the change in the patient state data satisfies the reference change, that the patient is experiencing pain. In addition, the method may include producing, by the compute device, a notification to another device that may be communicatively connected to the compute device. The notification may indicate that the patient is experiencing pain.

In some embodiments, the method may include obtaining, by the compute device, patient medication data from an intravenous pump associated with the patient. The method may also include determining, by the compute device and further as a function of the patient medication data, whether the patient is experiencing pain. In some embodiments, the method may include obtaining, by the compute device, image data indicative of a facial expression of the patient. The method may also include determining, by the compute device and further as a function of the obtained image data, whether the patient is experiencing pain.

The method, in some embodiments, may include obtaining, by the compute device, audio data that may be indicative of one or more sounds produced by the patient. Additionally, the method may include determining, by the compute device and further as a function of the obtained audio data, whether the patient is experiencing pain.

In another aspect of the present disclosure, one or more computer-readable storage media may include a set of instructions that, when executed, cause a compute device to obtain patient state data that may be indicative of a present state of a patient. The patient state data may include a heart rate of the patient and/or a respiration rate of the patient. Additionally, the instructions may cause the compute device to determine whether a change in patient state data satisfies a reference change. The instructions may also cause the compute device to determine, in response to a determination that the change in the patient state data satisfies the reference change, that the patient is experiencing pain. Further, the instructions may cause the compute device to produce a notification to another device that may be communicatively connected to the compute device, indicating that the patient is experiencing pain.

In some embodiments, the instructions may further cause the compute device to obtain patient medication data from an intravenous pump associated with the patient. The instructions may further cause the compute device to determine, further as a function of the patient medication data, whether the patient is experiencing pain. In some embodiments, the instructions may cause the compute device to obtain image data that may be indicative of a facial expression of the patient. Additionally, the instructions may cause the compute device to determine, further as a function of the obtained image data, whether the patient is experiencing pain. In some embodiments, the instructions may cause the compute device to obtain audio data that may be indicative of one or more sounds produced by the patient. The instructions may also cause the compute device to determine, further as a function of the obtained audio data, whether the patient is experiencing pain.

In another aspect of the disclosure, a pain prediction monitoring system may include one or more patient monitor devices that may obtain patient state data indicative of at least one of a patient's heart rate or respiration rate. The pain prediction monitoring system may also include a patient medication database that may include data indicative of a schedule for administration of pain medication to the patient. The pain prediction monitoring system may also include alerting circuitry that may be configured to determine whether a trend in the patient state data satisfies a predefined condition. The alerting circuitry may also be configured to determine whether the patient medication data indicates that the patient is due for administration of pain medication within a predefined time period. Additionally, the alerting circuitry may be configured to determine, in response to a determination that the trend in the patient state data satisfies the predefined condition and a determination that the patient is due for administration of pain medication within the predefined time period, that the patient is experiencing pain. Further, the alerting circuitry may be configure to send, in response thereto, an alert signal indicating that the patient is experiencing pain.

Additional features, which alone or in combination with any other feature(s), such as those listed above and/or those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a simplified diagram of at least one embodiment of a system for providing enhanced pain management;

FIG. 2 is a block diagram of at least one embodiment of components of a pain management compute device included in the system of FIG. 1;

FIGS. 3-7 are simplified flow diagrams of at least one embodiment of a method for providing enhanced pain management that may be performed by the system of FIG. 1.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

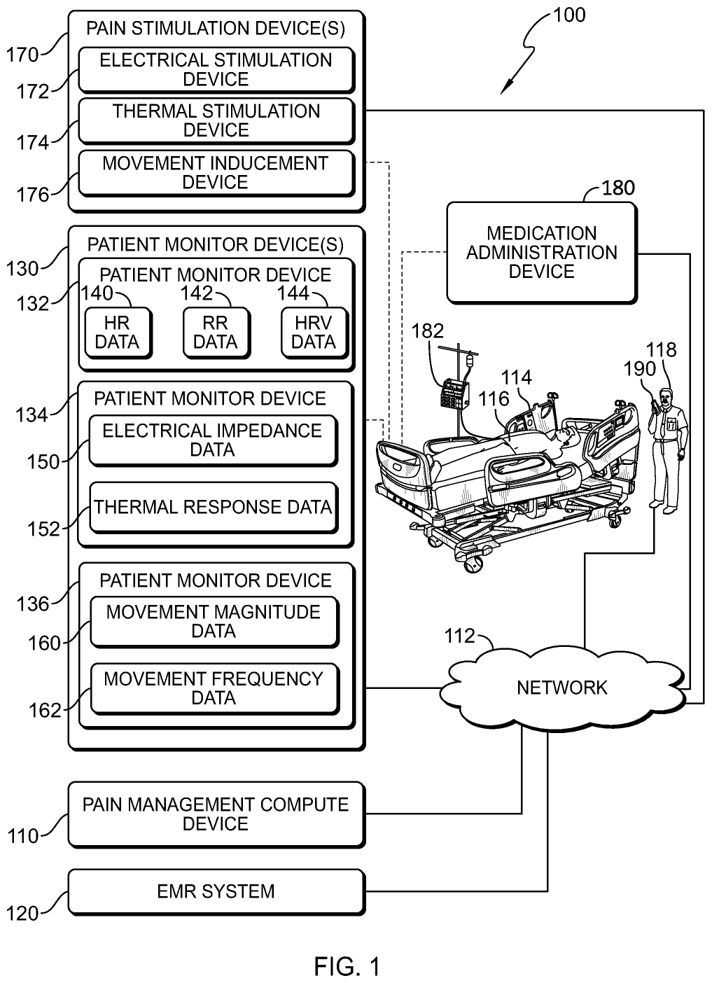

Referring now to FIG. 1, a system 100 for providing enhanced pain management includes a pain management compute device 110 in communication with an electronic medical records system 120, one or more patient monitor devices 130, one or more pain stimulation devices 170, a medication administration device 180, and a mobile compute device 190 of a caregiver 118 (e.g., a doctor, a nurse, etc.). The pain management compute device 110, in the illustrative embodiment, may be embodied as circuitry (e.g., alerting circuitry) that determines, based on information from the one or more patient monitor devices 130, whether a patient 116 is experiencing pain, thereby relieving caregivers from the burden of personally attempting to determine whether the patient 116 is in pain based on a subjective verbal report from the patient 116 or contextual information regarding the patient (e.g., in situations in which the patient is non-verbal). The pain management compute device 110 may send a notification to a caregiver (e.g., the caregiver 118) indicating that the patient 116 is in pain and/or may send a command to a medication administration device 180 (e.g., any device or circuitry configured to deliver medication to the patient 116, such as an intravenous pump 182, a drug delivery patch, etc.). In making the determination as to whether the patient is experiencing pain, the pain management compute device 110 may utilize information from the electronic medical records system 120 (e.g., any compute device or set of compute devices configured to store and provide electronic medical record data on request) to determine when the patient last received pain medication and when the patient is due for another dose of pain medication. Additionally or alternatively, in some embodiments, the pain management compute device 110 may obtain, from another source such as the intravenous pump 182, information indicative of when the patient last received pain medication and when the patient is due for another dose of pain medication. In some embodiments, the intravenous pump 182 may report to the pain management compute device 110 that the patient's medicine has run out and needs to be replenished. Relatedly, the intravenous pump 182 may report when the medicine has been replaced. As such, the pain management compute device 110 may utilize the information reported by the intravenous pump 182 in determining the present state of the patient and predicting a future state of the patient, including whether the patient is in pain, is in danger of experiencing opioid induced respiratory distress, etc.

In some embodiments, the pain management compute device 110 may utilize information from a pain stimulation process, in which one or more pain stimuli are applied to the patient (e.g., using the pain stimulation device(s)) 170 and a corresponding patient response is detected (e.g., using the patient monitor device(s) 130) to establish an objective measure of pain sensitivity of the patient 116. Additionally, the pain management compute device 110 may determine whether the patient is experiencing opioid induced respiratory distress and perform a corrective action, such as notifying a caregiver (e.g., the caregiver 118) well before the respiratory distress would otherwise be detected in a conventional system, as described in more detail herein. By providing the above features, which are described in more detail herein, the system 100 provides a more objective determination as to whether a patient (e.g., the patient 116) is in pain compared to traditional systems, may inform one or more caregivers and/or the patient of the amount of pain to expect under various circumstances, and may perform operations to manage the patient's level of pain, including controlling the administration of pain medication while guarding against opioid induced respiratory distress.

In the illustrative embodiment, the patient monitor devices 130 include a patient monitor device 132 which may be embodied as any device or set of devices or circuitry capable of collecting heart rate data 140 (e.g., any data indicative of the heart rate of the patient 116 over time), respiration rate data 142 (e.g., any data indicative of the respiration rate of the patient 116 over time), and, in at least some embodiments, heart rate variability data 144 (e.g., any data indicative of the variability in the heart rate of the patient 116 over time). In some embodiments, the patient monitor device 132 may additionally be capable of collecting movement data indicative of movements of the patient over time. The patient monitor device 132, in the illustrative embodiment, is a contact-free continuous monitoring device, such as an EarlySense CFCM device from Hill-Rom Holdings, Inc. of Batesville, Ind. The patient monitor device 132 may be located in or on a patient support apparatus, such as a patient bed (e.g., a Centrella® Smart+ Bed from Hill-Rom Holdings, Inc. of Batesville, Ind.), a chair, or other device capable of supporting the patient 116. In other embodiments, the patient monitor device 132 may be independent of the patient support apparatus, such as a wearable device (e.g., a respiration monitor belt capable of measuring expansion and contraction of a chest, a wristband with an integrated heart rate sensor, etc.).

The patient monitor device 134 may be embodied as one or more devices configured to measure changes in electrical and/or thermal impedance of the skin of the patient 116, such as a set of contacts on the patient's skin to measure an input electrical signal and an effect of electrical impedance of the patient's skin (e.g., the opposition, produced as a function of resistance and reactance, to the electrical signal) and/or a set of contacts on the patient's skin to measure an input thermal stimulus (e.g., heat) and an effect of thermal impedance of the patient's skin on the input thermal stimulus. Changes in the level of pain that the patient is presently experiencing can be correlated to a corresponding amount of impedance or a rate of change in the impedance. In the illustrative embodiment, the patient monitor device 134 produces electrical impedance data 150, which may be embodied as any data indicative of the electrical impedance of the patient's skin over time and thermal response data 152, which may be embodied as any data indicative of the thermal impedance of the patient's skin over time.

In the illustrative embodiment, the patient monitor device 136 is any device or circuitry configured to produce movement magnitude data 160, which may be embodied as any data indicative of the magnitude of one or more movements of the patient over time, and movement frequency data 162, which may be embodied as any data indicative of the frequency with which the patient has moved over time. As such, in some embodiments, the patient monitor device 136 may include a set of one or more load cells positioned underneath the patient (e.g., integrated into or placed on the patient support apparatus 114) to detect changes in the locations and amounts of force applied to the load cells due to movements of the patient 116 (e.g., rolling from one side of the patient support apparatus 114 to another side, lifting a limb to relieve pressure, etc.). In some embodiments, the patient monitor device 136 may include an image capture device (e.g., a video camera) directed at the patient 116 to identify changes in the position of the patient over time. In some embodiments, the image capture device may additionally or alternatively capture one or more images of the patient's face to be used in a facial recognition process to determine whether the patient 116 is in pain (e.g., grimacing, wincing, etc.). The patient monitor device 136, in some embodiments, may include a wearable device (e.g., a wrist band, ankle band, etc.) having an accelerometer configured to report changes in the direction and magnitude of acceleration, indicative of movement of the patient 116. In some embodiments, a microphone or other audio capture device may be present in the system 100 (e.g., in the patient support apparatus 114, integrated into the video capture device, etc.) to capture audio data from the patient 116, which may be indicative of whether the patient 116 is in pain (e.g., groaning, crying, moaning, words or phrases such as “I'm in pain,” “that hurts,” “ouch,” etc.).

The pain stimulation devices 170 may be embodied as any devices or circuitry capable of applying one or more stimuli to the patient to produce pain. As described in more detail here, by applying such stimuli, the system 100 (e.g., the pain management compute device 110) may determine a pain sensitivity of the particular patient 116 (e.g., as distinguished from another patient who may have a different pain sensitivity due to biological differences, such as hormonal levels, underlying health conditions, etc.). That is, by applying an objectively measured amount of stimuli and objectively measuring the physiological response of the patient 116 (e.g., with one or more of the patient monitor devices 130, such as changes in electrical or thermal impedance, changes in heart rate, etc.), the system 100 (e.g., the pain management compute device 110) may determine an objective baseline from which to determine how much pain the patient will feel in other situations, such as physical therapy sessions in which the patient is requested to perform certain movements known to produce discomfort, movements that the patient would be expected to make if released from bed rest at a particular time during the recovery period from surgery, etc. In the illustrative embodiment, the pain stimulation devices 170 includes an electrical stimulation device, which may be embodied as any device or circuitry configured to apply an electrical signal (e.g., a defined voltage at a particular frequency, etc.) to the patient 116 (e.g., to the skin of the patient 116). Additionally, in the illustrative embodiment the pain stimulation devices 170 include a thermal stimulation device 174, which may be embodied as any device or circuitry configured to apply a thermal stimulus (e.g., heat) to the skin of the patient 116. Further, in the illustrative embodiment, the pain stimulation devices 170 include a movement inducement device 176 which may be embodied as any device (e.g., an electromechanical device) or circuitry configured to move the patient's body through a defined range of motion (e.g., a range of motion that is known to induce pain in a physical therapy program).

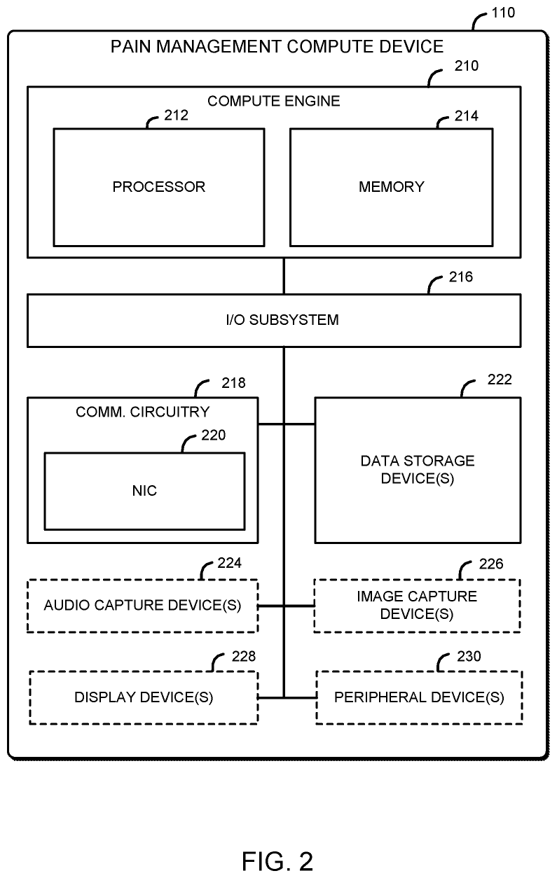

Referring now to FIG. 2, the illustrative pain management compute device 110 includes a compute engine 210, an input/output (I/O) subsystem 216, communication circuitry 218, and one or more data storage devices 222. The pain management compute device 110 may additionally include one or more audio capture devices 224, one or more image capture devices 226, one or more display devices 228, and/or one or more peripheral devices 230. Additionally, in some embodiments, one or more of the illustrative components may be incorporated in, or otherwise form a portion of, another component.

The compute engine 210 may be embodied as any type of device or collection of devices capable of performing various compute functions described below. In some embodiments, the compute engine 210 may be embodied as a single device such as an integrated circuit, an embedded system, a field-programmable gate array (FPGA), a system-on-a-chip (SOC), or other integrated system or device. Additionally, in the illustrative embodiment, the compute engine 210 includes or is embodied as a processor 212 and a memory 214. The processor 212 may be embodied as any type of processor capable of performing the functions described herein. For example, the processor 212 may be embodied as a single or multi-core processor(s), a microcontroller, or other processor or processing/controlling circuit. In some embodiments, the processor 212 may be embodied as, include, or be coupled to an FPGA, an application specific integrated circuit (ASIC), reconfigurable hardware or hardware circuitry, or other specialized hardware to facilitate performance of the functions described herein.

The main memory 214 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the main memory 214 may be integrated into the processor 202. In operation, the main memory 214 may store various software and data such as rules by which to determine whether a patient is experiencing pain, one or more machine learning models, and/or data obtained from the patient monitor devices 130, the EMR system 120, and/or other devices 114, 170, 180, 190 in the system 100, applications, libraries, and drivers.

The compute engine 210 is communicatively coupled to other components of the pain management compute device 110 via the I/O subsystem 216, which may be embodied as circuitry and/or components to facilitate input/output operations with the compute engine 200 (e.g., with the processor 212 and the main memory 214) and other components of the pain management compute device 110. For example, the I/O subsystem 216 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 216 may form a portion of a system-on-a-chip (SoC) and be incorporated, along with one or more of the processor 212, the main memory 214, and other components of the pain management compute device 110, into the compute engine 210.

The communication circuitry 218 may be embodied as any communication circuit, device, or collection thereof, capable of enabling communications over a network between the pain management compute device 110 and another device 114, 120, 130, 132, 134, 136, 170, 172, 174, 176, 180, 190. The communication circuitry 218 may be configured to use any one or more communication technology (e.g., wired or wireless communications) and associated protocols (e.g., Wi-Fi®, WiMAX, Bluetooth®, cellular, Ethernet, etc.) to effect such communication.

The illustrative communication circuitry 218 includes a network interface controller (NIC) 220. The NIC 220 may be embodied as one or more add-in-boards, daughter cards, network interface cards, controller chips, chipsets, or other devices that may be used by the pain management compute device 110 to connect with another device 114, 120, 130, 132, 134, 136, 170, 172, 174, 176, 180, 190. In some embodiments, the NIC 220 may be embodied as part of a system-on-a-chip (SoC) that includes one or more processors, or included on a multichip package that also contains one or more processors. In some embodiments, the NIC 220 may include a local processor (not shown) and/or a local memory (not shown) that are both local to the NIC 220. In such embodiments, the local processor of the NIC 220 may be capable of performing one or more of the functions of the compute engine 210 described herein. Additionally or alternatively, in such embodiments, the local memory of the NIC 220 may be integrated into one or more components of the pain management compute device 110 at the board level, socket level, chip level, and/or other levels.

Each data storage device 222 may be embodied as any type of device configured for short-term or long-term storage of data such as, for example, memory devices and circuits, memory cards, hard disk drives, solid-state drives, or other data storage device. Each data storage device 222 may include a system partition that stores data and firmware code for the data storage device 222 and one or more operating system partitions that store data files and executables for operating systems. Each audio capture device 224 may be embodied as any device or circuitry (e.g., a microphone) configured to obtain audio data (e.g., human speech, nonverbal sounds, etc.) and convert the audio data to digital form (e.g., to be written to the memory 214 and/or one or more data storage devices 222). Each image capture device 226 may be embodied as any device or circuitry (e.g., a camera) configured to obtain image data from the environment (e.g., images of the patient 116, such as facial expressions of the patient 116, movement of the patient's body, etc.) and convert the visual data to digital form (e.g., to be written to the memory 214 and/or one or more data storage devices 222).

Each display device 228 may be embodied as any device or circuitry (e.g., a liquid crystal display (LCD), a light emitting diode (LED) display, a cathode ray tube (CRT) display, etc.) configured to display visual information (e.g., text, graphics, etc.) to a viewer (e.g., a caregiver or other user). Each peripheral device 230 may be embodied as any device or circuitry commonly found on a compute device, such as a keyboard, a mouse, or a speaker to supplement the functionality of the other components described above.

The devices 114, 120, 130, 132, 134, 136, 170, 172, 174, 176, 180, 190 may have components similar to those described in FIG. 2 with reference to the pain management compute device 110. The description of those components of the pain management compute device 110 is equally applicable to the description of components of the devices 114, 120, 130, 132, 134, 136, 170, 172, 174, 176, 180, 190. Further, it should be appreciated that any of the devices 114, 120, 130, 132, 134, 136, 170, 172, 174, 176, 180, 190 may include other components, sub-components, and devices, including those commonly found in computing devices and medical equipment, which are not discussed above in reference to the pain management compute device 110 and not discussed herein for clarity of the description. Further, while shown separately in FIG. 1, it should be understood that in some embodiments, one or more of the devices 114, 120, 130, 132, 134, 136, 170, 172, 174, 176, 180, 190 may be combined or integrated into a single device (e.g., compute device). Additionally, while the components of a compute device may be shown as being housed in a single unit (e.g., housing), it should be understood that the components may be distributed across any distance and/or may be embodied as virtualized components (e.g., using one or more virtual machines utilizing hardware resources located in one or more data centers).

In the illustrative embodiment, the devices 114, 120, 130, 132, 134, 136, 170, 172, 174, 176, 180, 190 are in communication via a network 112, which may be embodied as any type of wired or wireless communication network, including local area networks (LANs) or wide area networks (WANs), digital subscriber line (DSL) networks, cable networks (e.g., coaxial networks, fiber networks, etc.), cellular networks (e.g., Global System for Mobile Communications (GSM), Long Term Evolution (LTE), Worldwide Interoperability for Microwave Access (WiMAX), 3G, 4G, 5G, etc.), radio area networks (RAN), global networks (e.g., the internet), or any combination thereof, including gateways between various networks.

The need to understand the level of pain that a patient is experiencing and manage the pain properly exists in many contexts, including an operating room, an intensive care unit (ICU), medical-surgical (MedSurg) nursing unit, and home (e.g., when a patient has been discharged). For example, hospitals and health systems have been trying to mobilize patients earlier as the clinical evidence suggests that the practice reduces length of stay (LOS) and reduces costs. However, this practice (mobilizing patients earlier) may cause patients to deal with significantly more pain induced by exercise and mobility (PIEM) than before. As such, an improved capability to measure pain objectively and accurately would be beneficial in a multitude of settings help with appropriately dosing patients with drugs (e.g., pain medications), explain for caregivers where the patient pain sensitivity is, and what level they will experience during different phases of their recovery, including physical therapy.

As stated above, the way each person feels pain is unique. When the pain threshold is exceeded inadvertently during physical therapy or during a patient's own or caregiver assisted movements in or out of a bed (e.g., patient support apparatus 114), patients stop moving and develop psychological barriers that prevent them from recovering expediently. Sometimes patients may spiral into recurring loop chronic pain, depression, and immobility. Poor management of opioids for such patients further aggravates the situation by making patients dependent on such drugs to function properly. Non-drug-based pain medication modalities (e.g., massage, patient education and preparation, meditation, acupuncture, etc.) exist but their application (intensity, frequency, etc.) can be significantly aided by knowledge of their predictive and actual effectiveness (e.g., through objective assessment of the patient's pain sensitivity and actual pain level).

Objectively understanding the pain sensitivity and starting pain of a patient allows customization of their exercise and mobilization protocols, in addition to helping to make informed decisions as to whether a painkiller prescription is justified together with the associated type and minimum needed dosage to enable execution of a prescribed therapy. Such an understanding is significant given that pain, whether it is chronic or induced by movement, is a major contributor to patient falls, compounding on top of existing patient fall risks. Pain also contributes to an additional length of stay (LOS), risk of readmission, and use of opioids after discharge. As the patient's predicted pain sensitivity and actual pain level inform the instantaneous and aggregate pain burden, it is possible to train and prepare the patient in advance to what is expected and what would be a normal level of pain for a given situation. Moreover, the capability to provide real-time objective pain information (e.g., a pain score) enables a physical therapist, occupational therapist, or other caregiver to know when to stop or decide to push further with a particular therapy. The management of the pain induced by exercise and mobility (PIEM) in this fashion may further decrease the risk of patient falls.

In addition to assisting with predicting PIEM and expediting patient recovery, the system 100, in the illustrative embodiment, utilizes data from the patient monitor devices 130, such as the heart rate data 140 and the respiration rate data 142, as well as electronic medical record data (e.g., patient medication data within the electronic medical record data) to determine whether a patient is presently experiencing pain and may be due for administration of pain medication. Additionally, the system 100, in operation, determines whether a patient is experiencing opioid induced respiratory distress based on a detected trend or decrease in respiration rate prior to when an alert would be triggered in a typical system, thereby giving caregivers more time to take a corrective action. In some embodiments, the system 100 may determine whether a patient is experiencing other negative effects from medication, based on the heart rate and/or respiration rate of the patient. For example, the intravenous administration of vancomycin can cause two types of reaction: (1) red man syndrome and (2) anaphylaxis (allergic reaction). Other drugs (e.g., ciprofloxacin, amphotericin, etc.) that stimulate histamine release can also result in this syndrome. Incidence is 3% to 47% of patients and it typically impacts patients 40 years old and younger. The syndrome can be magnified especially if patients are receiving vancomycin and other histamine stimulating drugs.

Red man syndrome, also referred to as vancomycin flushing syndrome, is typically related to the rapid infusion of the first dose of vancomycin. The side effects are a red rash and itchiness. Hypotension and dyspnea can occur depending on the severity of the case. The reaction can occur immediately or it can occur at the end of the infusion (e.g., after 60 minutes or, in some cases, after 90 to 120 minutes). The syndrome typically occurs during the first dose but can happen, albeit less often, on the seventh day of drug administration. As it relates to signs of anaphylaxis, skin reactions, low blood pressure, constriction of airways (e.g., difficulty breathing), and/or a weak but rapid pulse may occur.

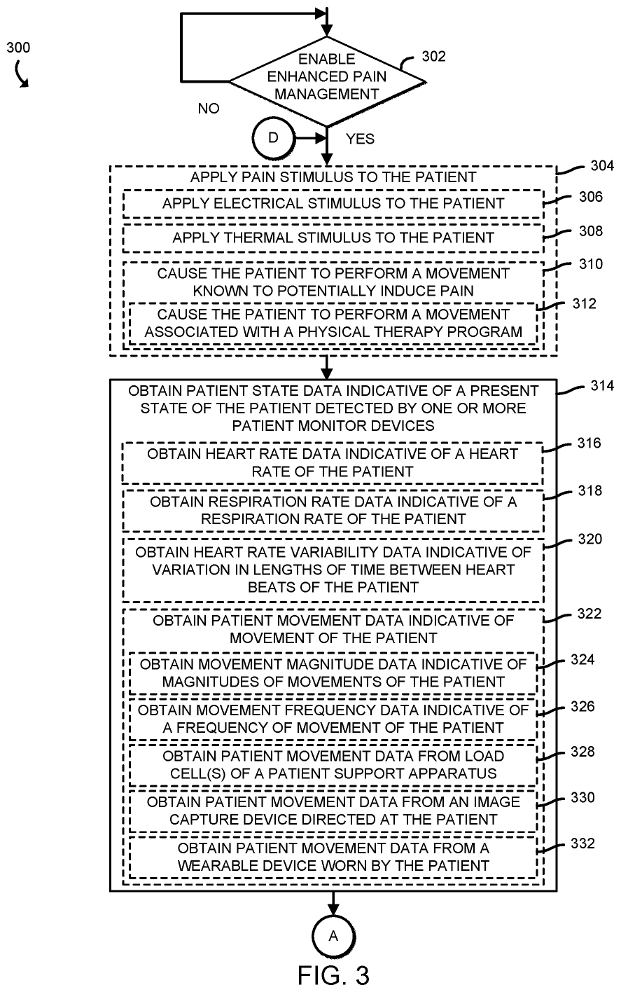

Referring now to FIG. 3, the system 100 (e.g., as controlled by the pain management compute device 110), may perform a method 300 for providing enhanced (e.g., compared to typical systems) pain management. In the illustrative embodiment, the method 300 begins with block 302, in which the pain management compute device 110 determines whether to enable enhanced pain management. In doing so, the pain management compute device 110 may determine to enable enhanced pain management in response to a determination that a configuration setting (e.g., in the memory 214 or data storage 222) indicates to do so, in response to a request (e.g., from the mobile compute device 190 of the caregiver 118), and/or based on other factors. Regardless, in response to a determination to enable enhanced pain management, the method 300 advances to block 304, in which the pain management compute device 110 applies (e.g., by sending a corresponding request through the network 112, a local bus connection, or other communication channel, to one or more of the pain stimulation device(s) 170) a pain stimulus to a patient (e.g., the patient 116), usable to determine the patient's physiological response to pain. In doing so, the pain management compute device 110 may apply (e.g., via a request to the electrical stimulation device 172) an electrical stimulus to the patient, as indicated in block 306. For example, the pain management compute device 110 may send a request to the electrical stimulation device 172 to apply an electrical signal having a defined voltage and frequency (e.g., in the case of an alternating current). In some embodiments, the pain management compute device 110 applies (e.g., via a request to the thermal stimulation device 174) a thermal stimulus to the patient, as indicated in block 308. In doing so the pain management compute device 110 may cause a portion of the thermal stimulation device 174 (e.g., a contact) to reach a defined temperature.

Additionally or alternatively, the pain management compute device 110 may cause the patient to perform a movement known to potentially induce pain (e.g., by presenting, on the display device 228 or through another output device, an instruction to the patient 116 to perform the movement, by sending a request to a caregiver to cause the patient 116 to perform the movement, by sending a request through the network 112 to the movement inducement device 176 to cause the patient 116 to perform a defined movement, etc.), as indicated in block 310. In doing so, the pain management compute device 110 may cause the patient 116 to perform a movement associated with a physical therapy program (e.g., by determining from electronic medical record data in the EMR system 120 that the patient 116 is or will be enrolled in a particular physical therapy program and by identifying, in a data set, one or more motions that the patient 116 will be expected to perform in connection with the physical therapy program), as indicated in block 312.

In block 314, the pain management compute device 110 obtains patient state data (e.g., via data transfer through the network 112, through a local bus between the pain management compute device 110 and the patient monitor devices 130, etc.) indicative of a present state of the patient 116 (e.g., resting in the patient support apparatus 114, as shown in FIG. 1, seated, standing, performing a movement, exercising, potentially receiving pain stimulation from a pain stimulation device 170, etc.) detected by one or more of the patient monitor devices 130. As explained in more detail herein, the patient state data may be subsequently utilized to determine whether the patient 116 is in pain, whether the patient 116 is experiencing opioid induced respiratory distress, and/or to establish a baseline level of pain sensitivity of the patient 116.

In obtaining the patient state data, and as indicated in block 316, the pain management compute device 110 may obtain heart rate data 140 (e.g., from the patient monitor device 132), which may be embodied as any data indicative of the heart rate of the patient over time. The pain management compute device 110, in the illustrative embodiment, also obtains respiration rate data 142 (e.g., from the patient monitor device 134), which may be embodied as any data indicative of the respiration rate of the patient 116 over time, as indicated in block 318. In some embodiments, as indicated in block 320, the pain management compute device 110 may also obtain heart rate variability data 144 (e.g., from the patient monitor device 130), which may be embodied as any data indicative of variation in the lengths of time between heart beats of the patient 116. As indicated in block 322, the pain management compute device 110 obtains movement data (e.g., from the patient monitor device 136) indicative of movement of the patient 116. In doing so, the pain management compute device 110 may obtain movement magnitude data 160, which may be embodied as any data indicative of magnitudes (e.g., a distance of the movement, a range of motion, a speed of the movement, etc.) of movements of the patient 116, as indicated in block 324. Relatedly, the pain management compute device 110 may obtain movement frequency data 162, which may be embodied as any data indicative of the frequency of the movements of the patient 116, as indicated in block 326.

As indicated in block 328, the pain management compute device 110 may obtain patient movement data from load cell(s) of a patient support apparatus (e.g., load cells positioned underneath the patient 116 in the patient support apparatus 114), in which changes in load over time at different locations are indicative of the frequency and magnitude of the movements of the patient 116. In some embodiments, the pain management compute device 110 may obtain patient movement data from an image capture device directed at the patient 116, as indicated in block 330. The pain management compute device 110, as indicated in block 332, may additionally or alternatively obtain patient movement data from one or more wearable devices worn by the patient 116, such as wrist band(s), ankle band(s), etc. having accelerometers configured to determine and report directions and magnitudes of their acceleration over time.

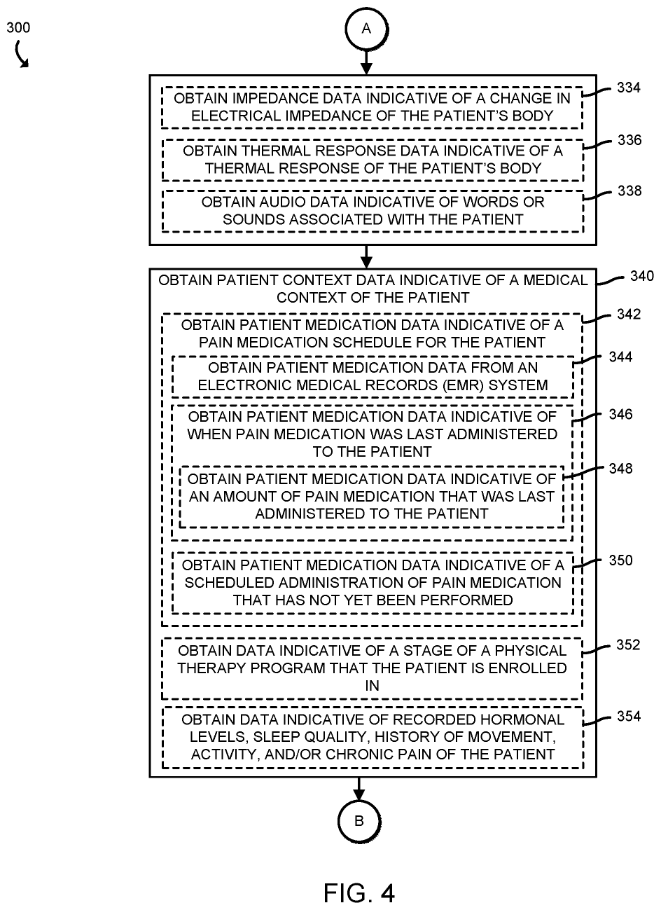

As discussed above, stresses on the human body, including pain, cause changes in the body's electrical and thermal impedance. As such, and referring now to FIG. 4, in collecting the patient state data, the pain management compute device 110 may obtain impedance data (e.g., the electrical impedance data 150) indicative of a change in electrical impedance of the patient's body, as indicated in block 334. Additionally or alternatively, and as indicated in block 336, the pain management compute device 110 may obtain thermal response data (e.g., the thermal response data 152) indicative of a thermal response of the patient's body. The pain management compute device 110 may also obtain audio data indicative of words or sounds associated with the patient 116 (e.g., the patient 116 stating that the patient 116 is experiencing pain, a non-verbal vocalization, such as a groan, etc.), as indicated in block 338. Additionally or alternatively, the pain management compute device 110 may obtain image data indicative of the present state of the patient 116 (e.g., one or more images of a facial expression of the patient, such as grimacing, wincing, etc. indicating that the patient 116 is in pain). In addition to obtaining the patient state data, the pain management compute device 110, in the illustrative embodiment, also obtains patient context data indicative of a medical context of the patient 116, as indicated in block 340. In obtaining the patient context data, the pain management compute device 110 may obtain patient medication data indicative of a pain medication schedule for the patient 116, as indicated in block 342. In the illustrative embodiment, the pain management compute device 110 obtains the patient medication data from the electronic medical records (EMR) system 120, as indicated in block 344.

The pain management compute device 110 obtains, in the illustrative embodiment, patient medication data that is indicative of when pain medication (e.g., an opioid-based medication) was last administered to the patient 116 as indicated in block 346 and, as indicated in block 348, further obtains patient medication data that is indicative of the amount of pain medication that was last administered to the patient 116 (e.g., at the time indicated in the data from block 346). Relatedly, and as indicated in block 350, the pain management compute device 110 may obtain patient medication data that is indicative of the a schedule administration of pain medication that has not yet been performed (e.g., when the next dose of pain medication is due). In other embodiments, the pain management compute device may obtain the patient medication data from another source, such as the intravenous pump 182.

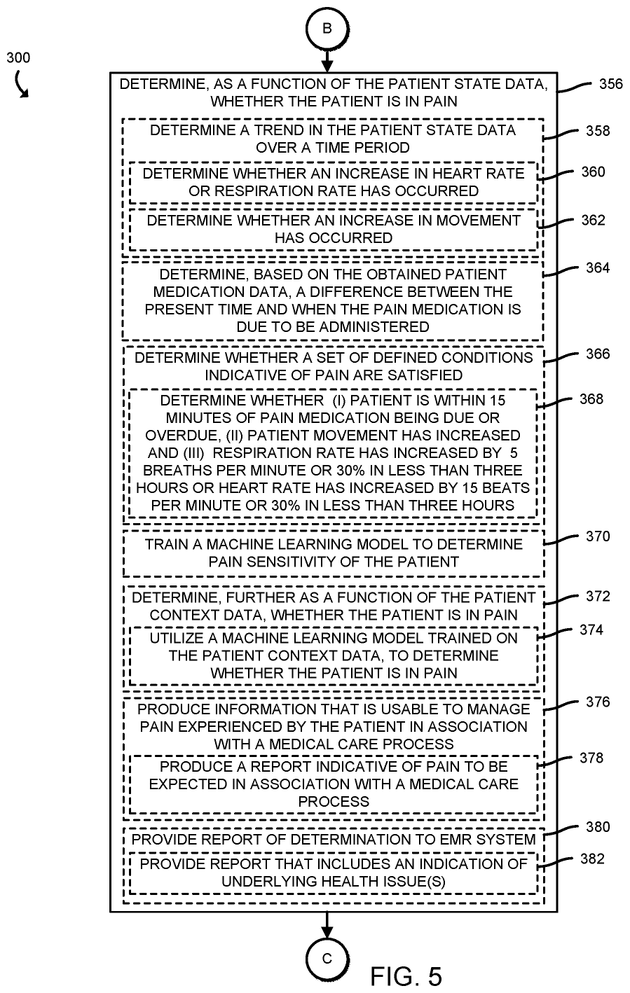

Aside from the medication information regarding the patient 116, the context of the patient may include other information. Accordingly, and as indicated in block 352, the pain management compute device 110 may obtain (e.g., from the EMR system 120) data indicative of a stage of a physical therapy program that the patient 116 is presently enrolled in. Further, in some embodiments, the pain management compute device 110 may obtain (e.g., from the EMR system 120) data indicative of recorded hormonal levels, sleep quality, history of movement, activity, and/or chronic pain experienced by the patient 116, as indicated in block 354. Subsequently, the method 300 advances to block 356 of FIG. 5 in which the pain management compute device determines, as a function of (e.g., based at least in part on) the patient state data, whether the patient 116 is in pain.

Referring now to FIG. 5, in determining whether the patient 116 is in pain, the pain management compute device 110 may determine a trend in the patient state data over a defined time period, as indicated in block 358. As indicated in block 360, the pain management compute device 110 determines whether an increase in heart rate or respiration rate has occurred. As indicated in block 362, the pain management compute device 110 may also determine whether an increase in movement (e.g., in magnitude and/or frequency) of the patient 116 has occurred. Regarding pain medication, the pain management compute device 110 may also determine, based on the obtain patient medication data (e.g., from block 346) a difference between the present time and when the pain medication is due to be administered, as indicated in block 364. In block 366, the pain management compute device 110 may determine whether a set of defined conditions indicative of pain are satisfied. In doing so, and as indicated in block 368, the pain management compute device 110 may determine whether the patient 116 is within 15 minutes of the pain medication being due or overdue (e.g., based on the determination from block 364), whether patient movement has increased (e.g., based on the determination from block 362), and whether the respiration rate of the patient 116 has increased by five breaths per minute or 30% (from an initial rate) in less than three hours or if the heart rate has increase by 15 beats per minute or 30% in less than three hours. That is, the pain management compute device 110 determines whether the above conditions regarding the timing of the pain medication and the movement of the patient are true and whether at least one of the conditions regarding the respiration rate or heart rate of the patient is true. In some embodiments, the pain management compute device 110 may utilize the patient state data and the patient context data to train a machine learning model (e.g., in the memory 214 or data storage 222 of the pain management compute device 110) to determine a pain sensitivity of the patient 116 (e.g., correlating a determination that the patient 116 is experiencing pain under the present conditions, which may include a pain stimulus, to set a baseline level of pain sensitivity of the patient 116), as indicated in block 370.

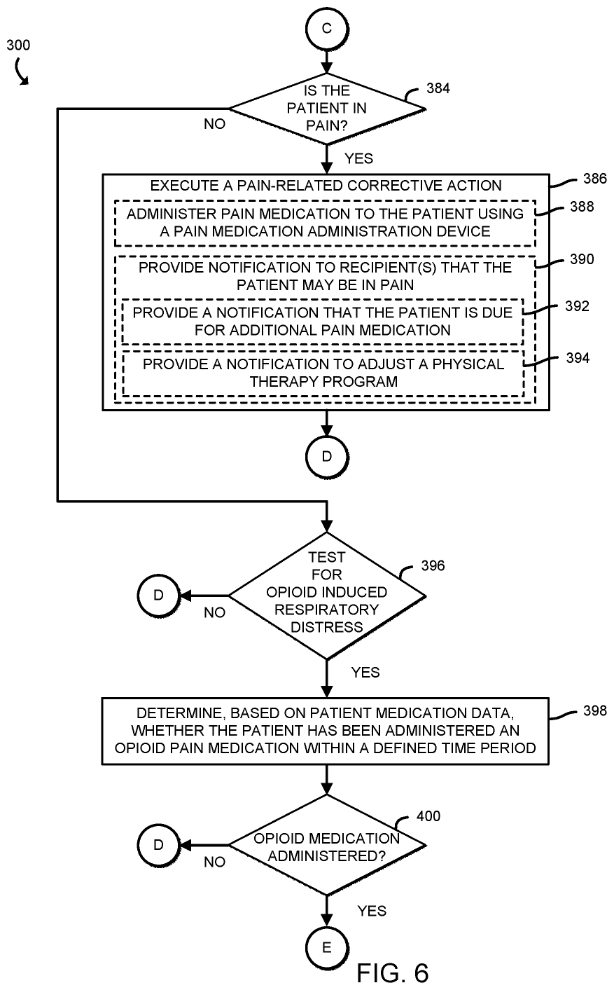

Still referring to FIG. 5, the pain management compute device 110 may determine whether the patient 116 is experiencing pain based not only on the patient state data but also the patient context data (e.g., from block 340 of FIG. 4), as indicated in block 372. In embodiments in which the pain management compute device 110 utilizes machine learning, the pain management compute device 110 may utilize a machine learning model (e.g., in the memory 214 and/or the data storage 222) trained on the patient context data, to determine whether the patient 116 is experiencing pain, as indicated in block 374. The pain management compute device 110, as indicated in block 376, may further produce information that is usable (e.g., capable of being rendered in a format that can be understood by a human, such as text, charts, graphics, etc. and/or usable by a device to adjust one or more operations, such as administration of pain medication) to manage pain that is presently experienced or that will be experienced by the patient 116 in association with a medical care process. In doing so, the pain management compute device 110 may adjust a set of predefined standard levels of pain to be expected in various activities associated with the medical care process based on the determined pain sensitivity of the patient 116 (e.g., increasing the expected pain if the patient's pain sensitivity is relatively high, decreasing the expected pain if the patient's pain sensitivity is relatively low, etc.). As indicated in block 378, the pain management compute device 110 may produce a report (e.g., a visual representation, audible representation, or other representation perceivable by a human) of pain to be expected in association with the medical care process. Relatedly, the pain management compute device 110 may provide a report of the determination (e.g., indicative of whether the patient 116 is experiencing pain, the patient's pain sensitivity, pain to be expected in the medical care process, etc.) to the EMR system 120, as indicated in block 380. In doing so, and as indicated in block 382, the pain management compute device 110 may provide a report that includes an indication of one or more underlying health issues associated with the patient 116 (e.g., a determination that the patient has fibromyalgia, etc.). Afterwards, the method 300 advances to block 384 of FIG. 6, in which the pain management compute device 110 determines a subsequent course of action based on whether the patient 116 is in pain (e.g., as determined by the pain management compute device 110).

Referring now to FIG. 6, if the patient 116 is in pain, the method 300 advances to block 386, in which the pain management compute device 110 executes a pain-related corrective action. In doing so, and as indicated in block 388, the pain management compute device 110 may administer pain medication to the patient 116 using a pain medication administration device (e.g., the medication administration device 180). In doing so, the pain management compute device 110 may send a request or other signal to the pain medication administration device 180 (e.g., through the network 112, through a local bus connection, etc.) to administer a defined amount of pain medication to the patient 116. Additionally or alternatively, the pain management compute device 110 may provide a notification (i.e., produce an alert signal) to one or more recipients (e.g., to the mobile compute device 190 of the caregiver 118) that the patient may be in pain (e.g., that the pain management compute device 110 has determined that the patient 116 is in pain), as indicated in block 390. The notification (alert signal) may be an audible alert, an alert on a screen, a nurse call signal, and/or a message to a caregiver mobile compute device 190. As indicated in block 392, the pain management compute device 110 may provide a notification indicating that the patient 116 is due for additional pain medication. Additionally or alternatively, as indicated in block 394, the pain management compute device 110 may provide a notification to adjust a physical therapy program (e.g., to reduce the amount of pain that the patient 116 is subjected to, in view of the patient's pain sensitivity and present amount of pain) or other medical process that the patient 116 is or will undergo. Subsequently, the method 300 loops back to block 304 to potentially repeat the operations of the method 300.

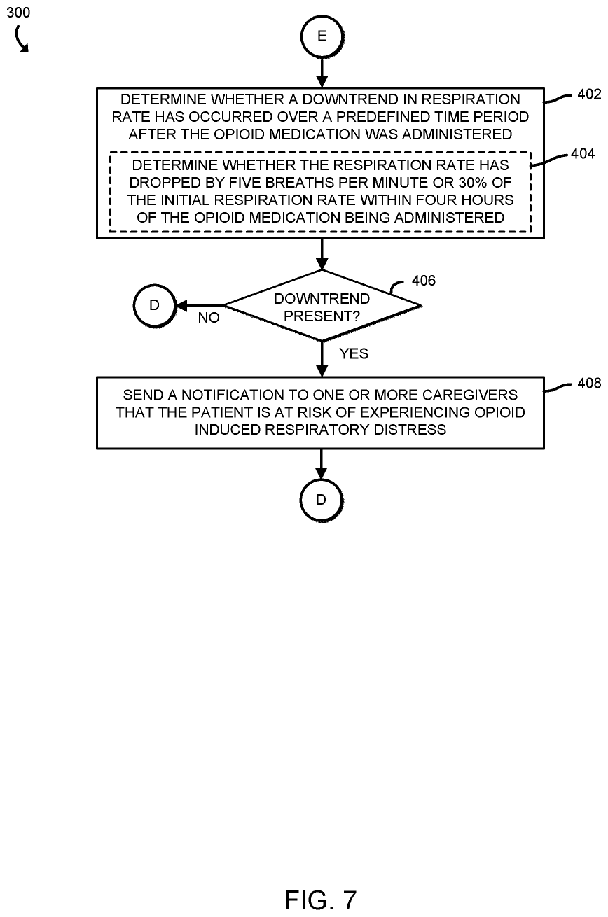

Referring back to block 384, in response to a determination that the patient 116 is not in pain, the method 300 advances to block 396 in which the pain management compute device 110 determines whether to test whether the patient 116 is in opioid induced respiratory distress. In the illustrative embodiment, the pain management compute device 110 determines to test for opioid induced respiratory distress unless a configuration setting (e.g., in the memory 214 or data storage 222) indicates not to. In response to a determination not to test for opioid induced respiratory distress, the method 300 loops back to block 302 of FIG. 3. Otherwise, the method 300 advances to block 398, in which the pain management compute device 110 determines, based on the patient medication data, whether the patient 116 has been administered an opioid pain medication within a defined time period (e.g., within the last four hours). As indicated in block 400, if the patient 116 has not received opioid medication within the defined time period, the method 300 loops back to block 302 of FIG. 3 to potentially repeat the operations of the method 300. Otherwise, the method 300 advances to block 402 of FIG. 7, in which the pain management compute device 110 determines whether a downtrend in respiration rate has occurred over a predefined time period (e.g., four hours) after the opioid medication was administered.

Referring now to FIG. 7, in determining whether a downtrend is present, the pain management compute device 110 may determine whether the respiration rate of the patient 116 has dropped by five breaths per minute or 30% of the initial respiration rate within four hours of the opioid medication being administered. In block 406, the pain management compute device 110 determines the subsequent course of action based on whether the downtrend is present. If not, the method 300 loops back to block 302 of FIG. 3 in which the operations of the method 300 are potentially executed again. Otherwise, the method 300 advances to block 408, in which the pain management compute device 110 sends a notification to one or more caregivers (e.g., to the mobile compute device 190 of the caregiver 118 and potentially to mobile compute devices of other caregivers and/or through one or more speakers or other devices capable of alerting caregivers of conditions), that the patient 116 is at risk of experiencing opioid induced respiratory distress. That is, in the illustrative embodiment, the pain management compute device 110 determines whether the patient 116 is on a trajectory to likely be in opioid induced respiratory distress in the next several hours, rather than detecting a present case of respiratory distress. As such, the pain management compute device 110 provides caregivers with far more notice (e.g., multiple hours more notice) than in typical systems that only detect respiratory distress that is already occurring.

Referring now to Table 1, in typical systems an alert is triggered when a patient's respiratory rates reaches an unsafe level of eight breaths per minute. At this point, the patient may already be in critical condition.

TABLE 1
Detection of Respiratory Distress
by a Conventional System at Hour 12

| Hour | Respiration Rate | Alarm Threshold |
|---|---|---|
| 1 | 18 | 8 |
| 2 | 18 | 8 |
| 3 | 17 | 8 |
| 4 | 16 | 8 |
| 5 | 15 | 8 |
| 6 | 14 | 8 |
| 7 | 13 | 8 |
| 8 | 12 | 8 |
| 9 | 11 | 8 |
| 10 | 10 | 8 |
| 11 | 9 | 8 |
| 12* | 8 | 8 |
| 13 | 8 | 8 |

By contrast, the illustrative pain management compute device 110, operating under the parameters described above with reference to block 404, provides a much earlier warning of opioid induced respiratory distress to caregivers, giving them ample time to take a corrective action. In Table 2 below, an opioid is given at hour 2. Respiration rate then steadily decreases every hour. At hour 8, the respiration rate has decreased by five breaths per minute, therefore triggering the alert. This is a full five hours before the default alert would trigger (e.g., at 8 breaths per minute) in a conventional system.

TABLE 2
Detection of Respiratory Distress by
the Illustrative System at Hour 7

| | | Opioid | | | % |
|---|---|---|---|---|---|
| Hour | RR | Given | Diff RR | Cum. Diff | Decrease |
| 1 | 18 | 0 | | | |
| 2 | 18 | 1 | 0 | 0 | 0% |
| 3 | 17 | 0 | −1 | −1 | −6% |
| 4 | 16 | 0 | −1 | −2 | −11% |
| 5 | 15 | 0 | −1 | −3 | −17% |
| 6 | 14 | 0 | −1 | −4 | −22% |
| 7* | 13 | 0 | −1 | −5 | −28% |
| 8 | 12 | 0 | −1 | −6 | −33% |
| 9 | 11 | 0 | −1 | −7 | −39% |
| 10 | 10 | 0 | −1 | −8 | −44% |
| 11 | 9 | 0 | −1 | −9 | −50% |
| 12 | 8 | 0 | −1 | −10 | −56% |
| 13 | 8 | 0 | 0 | −10 | −56% |

The parameters under which an alert may be triggered in the pain management compute device 110 may be reconfigured (e.g., overwritten in the memory 214 or storage 222) with other parameters, such as a decrease of 3 breaths per minute or 25% within five hours, to increase the sensitivity even further (e.g., thereby providing even more advanced notice to caregivers of the patient's condition). Furthermore, the pain management compute device 110 may be configured to detect a risk of and send a corresponding notification regarding other conditions associated with the administration of medication, such as vancomycin flushing syndrome or anaphylaxis, as discussed above. Regardless, after providing the notification in block 408, the method 300, in the illustrative embodiment, loops back to block 302 to potentially re-execute the operations of the method 300.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1. A compute device comprising:
circuitry configured to:
obtain patient state data indicative of a present state of a patient detected by one or more patient monitor devices, wherein the patient state data includes at least one of heart rate data indicative of a heart rate of the patient or respiration rate data indicative of a respiration rate of the patient;
obtain patient medication data indicative of a schedule for administration of pain medication to the patient;
determine whether a trend in the patient state data satisfies a predefined condition;
determine whether the patient medication data indicates that the patient is due for administration of pain medication within a predefined time period;
determine, in response to a determination that the trend in the patient state data satisfies the predefined condition and a determination that the patient is due for administration of pain medication within the predefined time period, that the patient is experiencing pain; and
produce, in response to a determination that the patient is experiencing pain, an alert signal.

2. The compute device of claim 1, wherein the circuitry is further configured to administer, in response to a determination that the patient is experiencing pain, pain medication to the patient using a pain medication administration device.

3. The compute device of claim 1, wherein to produce an alert signal comprises to produce an audible alert, an alert on a screen, a nurse call signal, or a message to a caregiver mobile device.

4. The compute device of claim 1, wherein the circuitry is further configured to:
determine a pain medication administration time indicative of when the patient was last administered pain medication;
determine a decline in the respiration rate of the patient over a predefined time period after the pain medication administration time; and
determine whether the decline satisfies a reference decline indicative of opioid induced respiratory distress.

5. The compute device of claim 4, wherein the circuitry is further configured to provide, in response to a determination that the decline satisfies the reference decline, a notification to a caregiver that the patient is experiencing opioid induced respiratory distress.

6. The compute device of claim 4, wherein to determine whether the decline in the respiration rate satisfies a reference decline comprises to determine whether the respiration rate has decreased by at least five breaths per minute or 30% of an initial respiration rate within a period of four hours after the pain medication administration time.

7. The compute device of claim 1, wherein to determine whether a trend in the patient state data satisfies a predefined condition comprises to determine whether the respiration rate of the patient has increased by five breaths per minute or 30% in less than three hours.

8. The compute device of claim 1, wherein to determine whether a trend in the patient state data satisfies a predefined condition comprises to determine whether the heart rate of the patient has increased by 15 beats per minute or 30% in less than three hours.

9. The compute device of claim 1, wherein to determine whether the patient medication data indicates that the patient is due for administration of pain medication within a predefined time period comprises to determine whether the patient is due for administration of pain medication within 15 minutes.

10. The compute device of claim 9, wherein the circuitry is further configured to:
obtain patient state data indicative of movement of the patient; and
wherein to determine whether a trend in the patient state data satisfies a predefined condition comprises to:
determine whether the movement of the patient has increased; and
determine whether the respiration rate of the patient has increased by five breaths per minute or 30% in less than three hours or the heart rate of the patient has increased by 15 beats per minute or 30% in less than three hours.

11. The compute device of claim 10, wherein to obtain patient state data indicative of movement of the patient comprises to obtain movement magnitude data indicative of magnitudes of movements of the patient and movement frequency data indicative of a frequency of movements of the patient; and
wherein to determine whether the movement of the patient has increased comprises to determine whether at least one of the magnitudes of the movements of the patient or the frequency of the movements of the patient has increased.

12. The compute device of claim 1, wherein to obtain patient state data indicative of movement of the patient comprises to obtain patient movement data from at least one of a set of load cells in a patient support apparatus, an image capture device directed at the patient, or a wearable device worn by the patient.

13. The compute device of claim 1, wherein to obtain patient state data comprises to obtain heart rate variability data indicative of lengths of time between heart beats of the patient.

14. A method comprising:
obtaining, by a compute device, patient state data indicative of a present state of a patient detected by one or more patient monitor devices, wherein the patient state data includes at least one of heart rate data indicative of a heart rate of the patient or respiration rate data indicative of a respiration rate of the patient;
obtaining, by the compute device, patient medication data indicative of a schedule for administration of pain medication to the patient;
determining, by the compute device, whether a trend in the patient state data satisfies a predefined condition;
determining, by the compute device, whether the patient medication data indicates that the patient is due for administration of pain medication within a predefined time period;
determining, by the compute device and in response to a determination that the trend in the patient state data satisfies the predefined condition and a determination that the patient is due for administration of pain medication within the predefined time period, that the patient is experiencing pain; and
producing, by the compute device and in response to a determination that the patient is experiencing pain, an alert signal.

15. The method of claim 14, further comprising administering, by the compute device and in response to a determination that the patient is experiencing pain, pain medication to the patient using a pain medication administration device.

16. The method of claim 14, wherein producing an alert signal comprises producing an audible alert, an alert on a screen, a nurse call signal, or a message to a caregiver mobile device.

17. The method of claim 14, further comprising:
determining, by the compute device, a pain medication administration time indicative of when the patient was last administered pain medication;
determining, by the compute device, a decline in the respiration rate of the patient over a predefined time period after the pain medication administration time; and
determining, by the compute device, whether the decline satisfies a reference decline indicative of opioid induced respiratory distress.

18. The method of claim 17, further comprising providing, by the compute device and in response to a determination that the decline satisfies the reference decline, a notification to a caregiver that the patient is experiencing opioid induced respiratory distress.

19. The method of claim 17, wherein determining whether the decline in the respiration rate satisfies a reference decline comprises determining whether the respiration rate has decreased by at least five breaths per minute or 30% of an initial respiration rate within a period of four hours after the pain medication administration time.

20. One or more computer-readable storage media comprising a plurality of instructions that, when executed, cause a compute device to:
obtain patient state data indicative of a present state of a patient detected by one or more patient monitor devices, wherein the patient state data includes at least one of heart rate data indicative of a heart rate of the patient or respiration rate data indicative of a respiration rate of the patient;
obtain patient medication data indicative of a schedule for administration of pain medication to the patient;
determine whether a trend in the patient state data satisfies a predefined condition;
determine whether the patient medication data indicates that the patient is due for administration of pain medication within a predefined time period;
determine, in response to a determination that the trend in the patient state data satisfies the predefined condition and a determination that the patient is due for administration of pain medication within the predefined time period, that the patient is experiencing pain; and
produce, in response to a determination that the patient is experiencing pain, an alert signal.
